SEQUENCE LISTING (1 sequence)

SEQ ID NO: 1 - protein, 11 aa, Lactobacillus plantarum YML007
IIVVGGIYRER

(19) United States
(12) Patent Application Publication
(10) Pub. No.: US 2017/0020141 A1
(43) Pub. Date: Jan. 26, 2017

(54) PEPTIDE HAVING FUNGICIDAL ACTIVITY, ISOLATED FROM LACTOBACILLUS PLANTARUM YML007

(71) Applicant: RESEARCH COOPERATION FOUNDATION OF YEUNGNAM UNIVERSITY, Gyeongsan-si, Gyeongsangbuk-do (KR)

(72) Inventors: Yong Ha PARK, Seoul (KR); Irfan Ahmad RATHER, Gyeongsan-si (KR); Byoung Joo SEO, Daegu (KR); Uk Han CHOI, Daegu (KR); Kwang Ho CHOI, Gyeongsan-si (KR)

(73) Assignee: RESEARCH COOPERATION FOUNDATION OF YEUNGNAM UNIVERSITY, Gyeongsan-si, Gyeongsangbuk-do (KR)

(21) Appl. No.: 15/039,420

(22) PCT Filed: Apr. 29, 2014
(86) PCT No.: PCT/KR2014/003799

§ 371 Date: Oct. 7, 2016

(30) Foreign Application Priority Data
Nov. 26, 2013 (KR) 10-2013-0144350

(51) Int. Cl. A01N 63/02; A23K 30/00; A23L 3/3571; C07K 14/335

(52) U.S. Cl. CPC A01N 63/02; C07K 14/335; A23K 30/00; A23L 3/3571; A23V 2002/00; A23Y 2220/67

(57) ABSTRACT

The present invention relates to a peptide having a fungicidal activity, isolated from Lactobacillus plantarum YML007, and provides a fungicidal peptide comprising the amino acid sequence of SEQ ID NO: 1. In addition, the present invention provides a fungicidal composition or a composition for food preservation, containing the peptide as an active ingredient. The peptide of the present invention has a wide range of fungicidal activities, and thus can be applied, by using the same, to various fields such as a microbial preparation related to fungicidal activity, an agent for controlling pathogenic fungi, a preservative for food including feed, and the future development of medical supplies related to fungi.

### TECHNICAL FIELD

The inventive concept relates to a peptide having a fungicidal activity, isolated from Lactobacillus plantarum YML007.

### BACKGROUND ART

Economical loss caused by plant pathogenic germs that are generated during culturing, transferring, or storing of agricultural crops has reached about 5 to 50%, where about 5 to 10% of the loss is caused by fungi, and the loss is further serious in the developing countries. Also, many studies have reported that damages caused by fungi include the secondary metabolic products produced by pathogenic microorganisms acting as a strong toxin to humans and animals and generation of fungal toxicosis in humans and livestock in addition to a simple reduction in harvest.

In hot and humid summer of Korea, environments appropriate for fungi to amplify in feeds may be formed. In particular, in the summer rainy season, a moisture content in feeds increases about 16%, and a relative humidity about 80 to 85%, and thus there may be rapid fungi growth. When fungi grows excessively in the feeds, various mycotoxins as the metabolic product may be produced, and the types and secretion amounts thereof may be modified by various causes such as types of fungi, temperature, humidity, and moisture contents of the feeds. However, even when the presence of mycotoxins in the feeds is at a trace amount, the damage on livestock may be significant, or the mycotoxins may be metastasized into human body through the livestock, and thus may have many problems. Currently, at least 400 mycotoxins have been identified, and an important mycotoxin group has been a main issue in terms of health of humans and animals, where aflatoxins, ochratoxin A, trichothecenes (deoxynivalenol and nivalenol), zearalenon, and fumonisins have been frequently observed in food and feeds.

Currently, there are methods of heating, ammonia-treating, screening, or radiating food or feeds, in order to suppress growth of fungi and generation of mycotoxins thereof, but industrial application of the methods is either too expensive or not practical, which may cause destruction of nutrition in food or feeds, and when a chemical prevention is performed by using a germicidal agent, side effects caused by the chemical prevention may occur.

Lactic acid bacteria is a bio-preserving microorganism, which has been used as a culture starter in food industry for long and has been known as capable of producing various types of bioactive molecules such as organic acids, fatty acids, hydrogen peroxide, and bacteriocin. Currently, various antifungal microorganisms have been discovered, but those cannot exhibit stable antifungal activity in a broad spectrum, and thus efforts to find antifungal microorganisms against various spoilage fungi have been continuously made. Lactic acid bacteria may be isolated in various environment such as grass silages, vegetables, or sourdough and thus may have an antifungal ability in a broad spectrum.

Kimchi is well-known lactic acid fermentation food which includes various types of lactic acid bacteria (LAB). The present inventors have isolated Lactobacillus plantarum YML007, which is a kimchi LAB having a strong fungicidal activity in a broad spectrum against various food spoilage fungi, and isolated a peptide that has a fungicidal activity of a broad spectrum from a strain to confirm the characteristics, thereby completing the present invention.

Meanwhile, Japanese Patent No. 1994-2714415 is related to a novel antibacterial material of lactic acid fungi and its combined material, which discloses a peptide having a structure of H-Arg-Pro-Lys-His-Pro-Ile-Lys-His-Gln-OH as an antifungal material that is a polymer material biosynthesized by bacteria, Lactobacillus helveticus K-4 (FERM-P 12249). However, there is no disclosure about a fungicidal peptide isolated from Lactobacillus plantarum YML007according to the present invention.

### DETAILED DESCRIPTION OF THE INVENTIVE CONCEPT

### Technical Problem

The inventive concept provides a fungicidal peptide comprising an amino acid sequence of SEQ ID NO: 1.

The inventive concept provides a fungicidal composition or a composition for food preservation, containing the peptide as an active ingredient.

### Technical Solution

According to an aspect of the inventive concept, there is provided a fungicidal peptide including an amino acid sequence of SEQ ID NO: 1.

The peptide may be derived from Lactobacillus plantarum YML007 (KCTC 12032BP).

The fungicidal peptide may be effective in controlling Aspergillus niger or Aspergillus flavus.

According to another aspect of the inventive concept, there is provided a fungicidal composition including the peptide as an active ingredient.

The fungicidal composition may be effective in controlling Aspergillus niger or Aspergillus flavus.

According to another aspect of the inventive concept, there is provided a composition for food preservation, wherein the composition contains the peptide as an active ingredient.

### Advantageous Effects

As described herein, an aspect of the inventive concept relates to a peptide having a fungicidal activity, isolated from Lactobacillus plantarum YML007, wherein the peptide comprising 11 amino acids and having a molecular weight of 1256.617 Da has a wide range of fungicidal activities, and thus can be applied, by using the same, to various fields such as a microbial preparation related to fungicidal activity, an agent for controlling pathogenic fungi, a preservative for food including feed, and the future development of medical supplies related to fungi.

### DESCRIPTION OF THE DRAWINGS

FIG. 1 illustrates a fungicidal activity of fractions isolated from A. niger and A. flavus. It was confirmed that the fungicidal activity was strongly suppressed after spore formation;

FIG. 2 shows a MALDI-TOF spectrum of active fractions purified from a Lactobacillus plantarum YML007 cell-free supernatant exhibiting a fungicidal activity;

FIG. 3 illustrates amino acid sequences of a fungicidal compound and measured and calculated weights thereof;

FIG. 4 shows the results of multiple sequence comparison using Multiple Sequence Comparison by Log-Expectation (MUSCLE);

FIG. 5 shows an average distance phylogenic tree using percentages. The phylogenic tree has been presented based on 12 protein sequences derived from microorganisms that are different from each other;

FIG. 6 shows (a) a cartoon secondary structure, (b) a wire frame secondary structure, and (c) a dot surface secondary structure of the fungicidal peptide isolated from Lactobacillus plantarum YML007; and

FIG. 7 illustrates sequence comparison between the fungicidal peptide isolated from Lactobacillus plantarum YML007 and other fungicidal materials isolated from another fungi in percentages.

### BEST MODE

The present invention provides a fungicidal peptide comprising an amino acid sequence of SEQ ID NO: 1. The SEQ ID NO: 1 is IIVVGGIYRER.

Particularly, the peptide is derived from Lactobacillus plantarum YML007 (KCTC 12032BP.

Also, the fungicidal peptide may be effective in controlling, but not limited to, at least one selected from the group consisting of yeast, mold fungi, filamentous fungi, rust fungi, and mushrooms, or, preferably, may be at least one microorganism selected from the group consisting of Aspergillus, Fusarium, Penicillium, Candida, and Saccharomyces. More preferably, the fungicidal peptide may be effective in controlling Aspergillus niger or Aspergillus flavus.

The present invention deems mutants of the sequence as included within the scope of the present invention in addition to the peptide comprising an amino acid sequence of SEQ ID NO: 1. Although base sequences or amino acid sequences of the mutants are different from those of the peptide according to the present invention, the mutants are peptides that comprise amino acid sequences, which are formed of base sequences having similar functional characteristics with those of the amino acid sequence of SEQ ID NO: 1. Particularly, the peptide according to the present invention may include an amino acid sequence having at least 70%, preferably, at least 80%, more preferably, at least 90%, or most preferably at least 95% sequence identity with the amino acid sequence of SEQ ID NO: 1.

Also, the present invention provides a fungicidal composition including the peptide as an active ingredient.

For example, the fungicidal composition may be used to suppress toxin production of fungi inhibiting in plants eaten by animals or livestock and the toxin production of the fungi may be suppressed by applying the fungicidal composition during sowing, cultivating, harvest, storing, and processing the plants, and before expatriation date of the plants.

The fungicidal composition of the present invention may be formulated in the form of a powder, a pellet or granule, or a microcapsule to include the peptide according to a general formulation method in the art and may be used as it is. The fungicidal composition may be obtained by adding other components such as surfactants, dispersants, surface-active agents, carriers, preservatives, moisturizers, supply-accelerators, attractants, capsulizers, binders, emulsifiers, dyes, U.V. protectors, and buffers that may be easily applied according to the subject of dispersion such as target pathogenic fungi, crops, and grains. Also, an excipient that is being additionally included may be, preferably, an appropriate material according to a use and an application method of the fungicidal composition, where the material may be, preferably, any material that is not harmful to plants or animals.

Also, the present invention provides a composition for food preservation, wherein the compound includes the peptide as an active ingredient.

Food in the present invention may denote any food that may be eaten by animals including insect, and the food may include feeds.

Examples, to which the composition for food preservation may be applied, may include grain processed product, vegetable, fruit, dried products or cut products, fruit juice, vegetable juice, mixed juice of vegetable and fruit, chips, noodles, livestock processed food products, marine processed food products, dairy products, fermented milk products, microbial fermented food, bakery and confectionery, spices, meat processed products, acidic drinks, licorices, herbs, insects feeds, livestock feeds, and pet feeds, but they are not limited thereto. The composition for food preservation has growth inhibition and extinction effects on harmful pathogenic microorganisms and thus may allow food to be maintained or preserved for a long time compared to that of a non-processed group.

The composition for food preservation may be variously formulated in the form of liquid, solid, or powder by using a convenient and appropriate method that corresponds the need of food preservation. The composition for food preservation may be assorted with a raw material during a food preparation process, and, in one embodiment of the present invention, the composition may be evenly distributed in food by immersing or stirring after immersing the food to the composition for food preservation or by spraying the composition for food preservation.

### MODE OF THE INVENTIVE CONCEPT

Hereinafter, the present invention will now be described with reference to examples to help understanding of the present invention. However, these examples set forth herein are provided for the purpose of illustration of the present invention, and the scope of the present invention is not limited to the examples.

Embodiments of the present invention will be described in detail with reference to the following examples. However, these examples are not intended to limit the scope of the present invention. These examples are provided so that this disclosure will fully convey the scope of the invention to those skilled in the art.

### Example 1

### Isolation of Fungicidal Peptide from Lactobacillus plantarum YML007

1. Antifungal Activity Assays

Lactobacillus plantarum YML007 was subjected to secondary screening using paper disk assay. Lactobacillus plantarum YML007 cultured in MRS broth at 30° C. for 24 hours was centrifuged at 10,000 g for 10 minutes and the cell-free supernatant was taken after filtering through a 0.45 μm pore-size filter (Sartorius Stedim Biotech, Germany). The pH of the supernatant was adjusted to 6 with 1 M NaOH and was used for further experiments. 100 μl of the supernatant was loaded on paper discs of 8 mm diameter (Advantec Roshi Kaisha, Ltd, Tokyo, Japan) on plates. The plates were incubated at 30° C. and examined after 24 hours and 48 hours for inhibition zones. The diameters of the inhibition zones were measured and the diameters of the 8 mm paper disk were subtracted from the total diameter of the inhibition zone. Lactobacillus plantarum YML007 was further analyzed for its broad fungicidal activity against other important spoilage molds Fusarium oxysporum KCTC6434, A. flavus KCTC16682, and A. oryzea KCCM 11371 using a paper disk method as described above.

2. Characterization of the Fungicidal Compound

The preliminary nature of the fungicidal compound was assessed by treating the culture filter of Lactobacillus plantarum YML007 with proteinase K (Sigma), trypsin (Sigma), lipase (Sigma), alpha amylase (Sigma), and catalase (Sigma), each at a final concentration of 1 mg/mL, and the solution was incubated at 37° C. for 3 hours. The mixtures were cooled to inactivate the enzyme activity and were subsequently analyzed by paper disc method for fungidical activity against A. niger KCTC16683. To further investigate the nature of the fungidical compound, 1 L of the cell-free supernatant of Lactobacillus plantarum YML007 was subjected to ammonium sulfate precipitation (80% w/v) followed by dialysis. The dialyzed sample was purified by gel filtration chromatography using Sephadex G-25 column (35×2 cm) with fraction ranges 1000-5000 kDa (Sigma Aldrich, USA). 20 mM phosphate buffer (pH 7) was used as an elusion buffer at a flow rate of 40 μl min⁻¹. A total of 28 fractions were collected and checked for fungicidal activity. The most active fraction was analyzed by MALDI-TOF (Ultra flex III, Bruker Daltonics, Germany) with α-cyano-4-hydroxy-cinnamic acid (HCCA) as matrix. A model VSL-337 ND S-nitrogen laser (337 nm) was used. The ion acceleration voltage and laser intensity were set to 20 kV and 35%, respectively. 5 μl of suspension of purified sample was carefully mixed with an equal volume of HCCA. 5 μl of the prepared solution was spotted onto a MALDI plate and left to dry at room temperature for 15 minutes before analysis.

3. Results

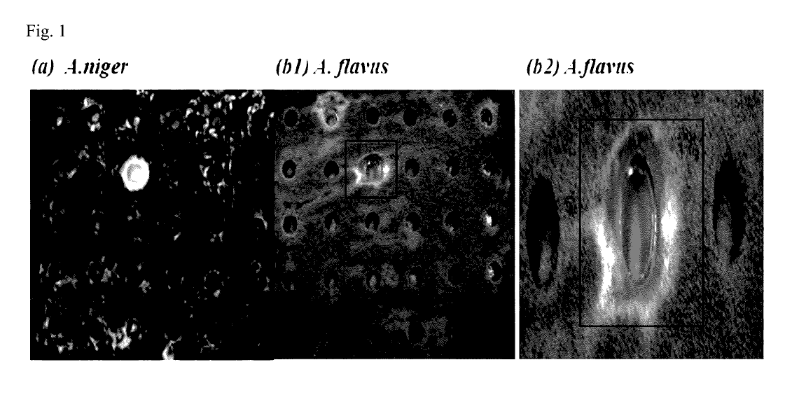

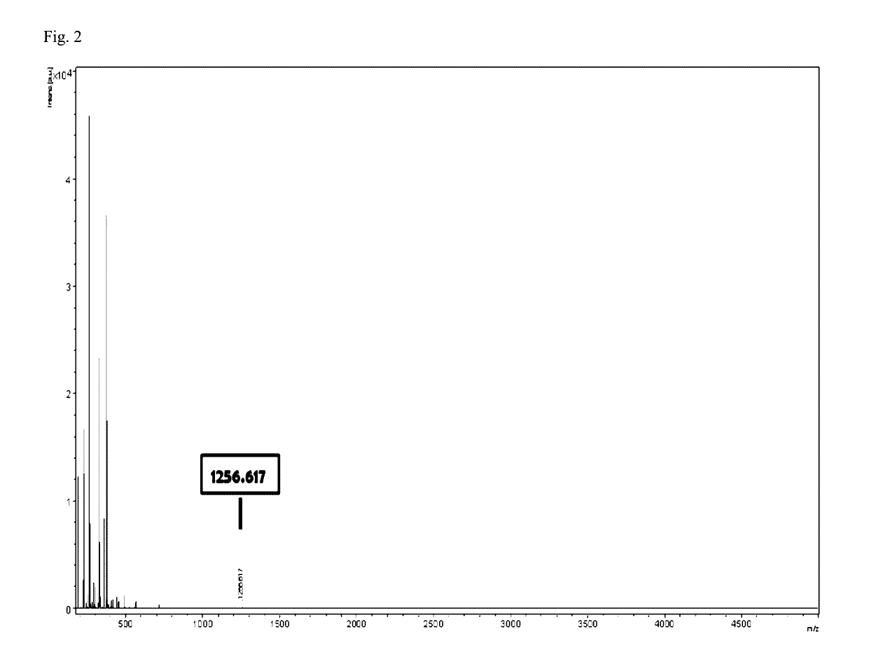

As the results of enzyme analysis, the fungicidal activity of the Lactobacillus plantarum YML007 was not affected by addition of catalase, and this indicates that the observed growth inhibition is not due to the hydrogen peroxide product. However, treatment of protein hydrolysis enzymes (protenase K and trypsin) removed the inhibitory activity of the cell-free supernatant, which indicates that the compound exhibits protein-like characteristics. The activity of the sample was not affected by alpha amylase and lipase, and this eliminates possibility of the active compound being lipid or carbohydrate. Partial isolation of the fungicidal compound was performed by ammonium sulfate precipitation, and the resultant was dialyzed by using a cellulose membrane (where a cut-off value was 3 kDa) to recover 85% of the activity after the filtration. From the results, it was estimated that a molecular weight of the compound may be lower than 3 kDa. The 28 fractions from the dialyzed sample were collected through a chromatography column. Among the 28 fractions, 9 fractions exhibited significant inhibitory activity. It was confirmed that the finally isolated fraction was a proteinous compound that is produced by Lactobacillus plantarum YML007, which exhibited the activity with the subject of the test fungi (FIG. 1). In the MALDI-TOF analysis, an active compound having a molecular weigh of 1256.617 Da was detected (FIG. 2)

### Example 2

### Characterization of the Fungicidal Peptide Isolated from Lactobacillus plantarum YML007

1. Strain and Growth Condition

In order to produce a fungicidal compound, Lactobacillus plantarum YML007 was incubated in an optimized MRS media. As indicator strains, Aspergillus niger and Aspergillus flavus were grown on potato dextrose agar (PDA) at 30° C. for 5 to 7 days.

2. Fungicidal Activity Analysis

The fungicidal activity of Lactobacillus plantarum YML007 selected as against various fungus germs and food-originated pathogenic germs was performed by using agar diffusion analysis. Lactobacillus plantarum YML007 cultured in MRS broth at 30° C. for 24 hours was centrifuged at 10,000 g for 10 minutes and the supernatant was taken after filtering through a 0.45 ml-pore-size filter (Sartorius Stedim Biotech, Germany). The pH of the supernatant was adjusted to 6 with 1 mol l⁻¹ NaOH and was used for further experiments. 100 μl of the supernatant was loaded in each well against F. oxysporum KCTC6434, A. flavus KCTC16682, and A. oryzae KCCM 11371 and food-originated pathogenic microorganisms E. coli KCTC2441, E. coli o157: H7, E. coli KCTC1039, Staphylococcus aureus KCTC1621, Staphylococcus epidermidis KCTC1917, Bacillus subtilis KCTC1021, Enterobacter cloacae KCTC2361, Klebsiella pneumoniae KCTC2208, L. monocytogenes KCTC3569, Salm. choleraesuis ATCC10727, Salmonella enterica ATCC4931, Cronobacter muytjensii ATCC51329, and Neisseria meningitides ATCC13098 on plates. The plates were incubated at 30° C. and examined after 24 hours and 48 hours for inhibition zones. The diameters of the inhibition zones were measured and the diameters of the 8 mm paper disk were subtracted from the total diameter of the inhibition zone.

3. Purification of the Fungicidal Compound

Purification was performed by a multi-step protocol. The supernatant enriched by ammonium sulfate precipitation followed by dialysis through a cellulose membrane with a cut-off at 3 kDa or lower. The active fractions were subsequently desalted by gel-filtration chromatography usingSephadex G-25 column (35×2 cm) with fraction ranges 1000-5000 MW (Sigma Aldrich). 20 mM phosphate buffer (pH 7) was used as an elution buffer at a flow rate of 40 μl min⁻¹. The most active fraction was further ultra-filtrated with Reversed-phase HPLC.

4. In-Gel Protein Digestion and Amino Acid Sequencing

Protein band of interest was excised and digested in-gel with sequencing grade, modified trypsin (Promega, Madison, Wis., USA). A target protein spot was excised from the gel, placed in a polypropylene (Eppendorf) tube, and washed 4 to 5 times until the gel was clear with 150 μl of 1:1 acetonitrile/25 mM ammonium bicarbonate, pH 7.8. The gel slice was dried in a Speedvac concentrator, and then rehydrated in 30 μl of 25 mM ammonium bicarbonate, pH 7.8, containing 20 ng of trypsin. After incubation at 37° C. for 20 hours, the liquid was transferred to a new tube. Tryptic peptides remaining in the gel matrix was extracted for 40 min at 30° C. with 20 μl of 50% (v/v) aqueous acetonitrile containing 0.1% (v/v) formic acid. The combined supernatants were evaporated in a Speedvac concentrator and dissolved in 8 μl of 5% (v/v) aqueous acetonitrile solution containing 0.1% (v/v) formic acid for mass spectrometric analysis.

5. Multiple Sequence Alignments

The amino acid sequence of antimicrobial peptide was run in BLAST and related sequences showing homology were aligned using Multiple Sequence Comparison by Log-Expectation (MUSCLE). The phylogenetic tree was constructed based on multiple sequence homologies.

6. Secondary Structure Prediction

GOR V server for protein secondary structure prediction was used for secondary structure prediction of antimicrobial peptide. The GOR V server is based on the database of Cuff and Barton of 513 sequentially non-redundant domains, which contains 84107 residues. After randomization, only the aligned sequences with a Z score <5 are considered dissimilar to hinder homology among sequences.

7. Results

(1) Amino Acid Sequencing

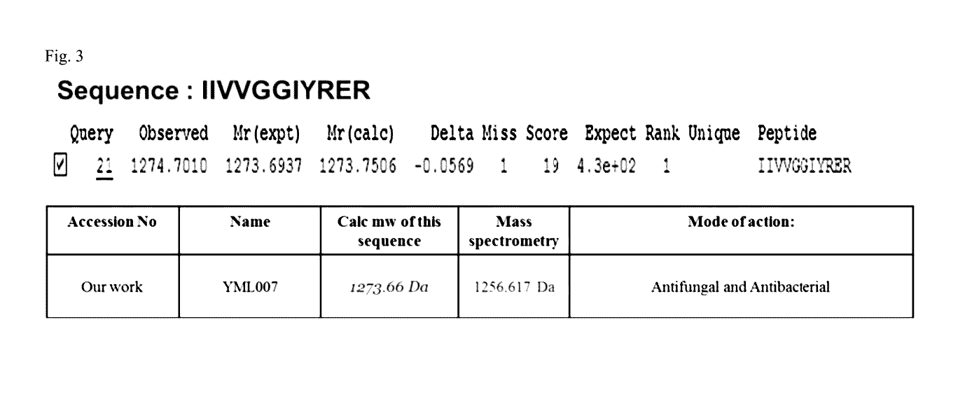

The amino acid sequencing by LC-MS/MS using nanoLC equipped QSTAR XL (ESI-Q-TOF) was found to be an 11 amino acid peptide with calculated molecular weight of 1273.66 Da. So, the difference of 17 was observed. It means that the peptide IIVVGGIYRER(SEQ ID NO: 1) has a calculated molecular weight of 1273.66 Da, where the previously obtained mass is equal to M-M (NH₃)=M-17, which fits previously observed mass 1256.617 Da. This means that there was a loss of M-17 during MALD-TOF analysis (FIG. 3).

The amino acid sequence was compared with the known entries in different data bases on the basis of protein-protein homology. The comparison showed that the amino acid sequenced residues have 30-40% homology with following known peptides (Table 1).

TABLE 1
Homology of YML007 with other antimicrobials

| Database | Compound name | Source |
|---|---|---|
| NCBInr | Nucleoside | Pseudomonas syringae pv. |
| | 2-deoxyribosyltransferase | lachrymans str.M301315 |
| | family protein | |
| The | Bactenecin (cyclic | Bovine neutrophils, |
| antimicrobial | dodecapeptide) activity | Bostaurus |
| peptide | against Gram + and | |
| database | Gram − bacteria | |
| The | Retrocyclin-1 (activity against | “Dead” gene-derived and |
| antimicrobial | virus, HIV) | synthetic |
| peptide | | |
| database | | |
| NCBInr | Alpha-galactosidase | Lb. plantarum |

(2) Classification of Antimicrobial Peptide

Based on its hydrophobic nature and broad range of antimicrobial activity, and source, the present inventors predicted that the peptide is related with Class II bacteriocins.

(3) Multiple Sequence Alignments

Multiple Sequence Alignments (MSA) is generally the alignment of three or more biological sequences (protein or nucleic acid) of similar length. From the compared homology, the evolutionary relationships between sequences can be inferred. Pairwise Sequence Alignment tools are used to identify regions of similarity that may indicate functional structure and/or evolutionary relationships between two biological sequences. However, it is difficult to align a small peptide with 10 to 15 amino acids with a protein having 50 to 300 amino acids.

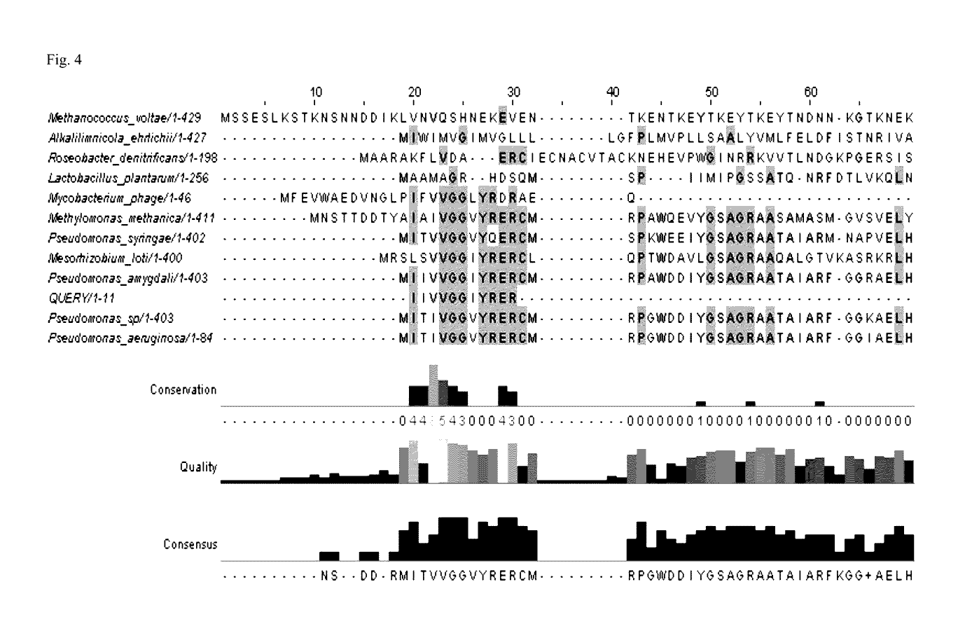

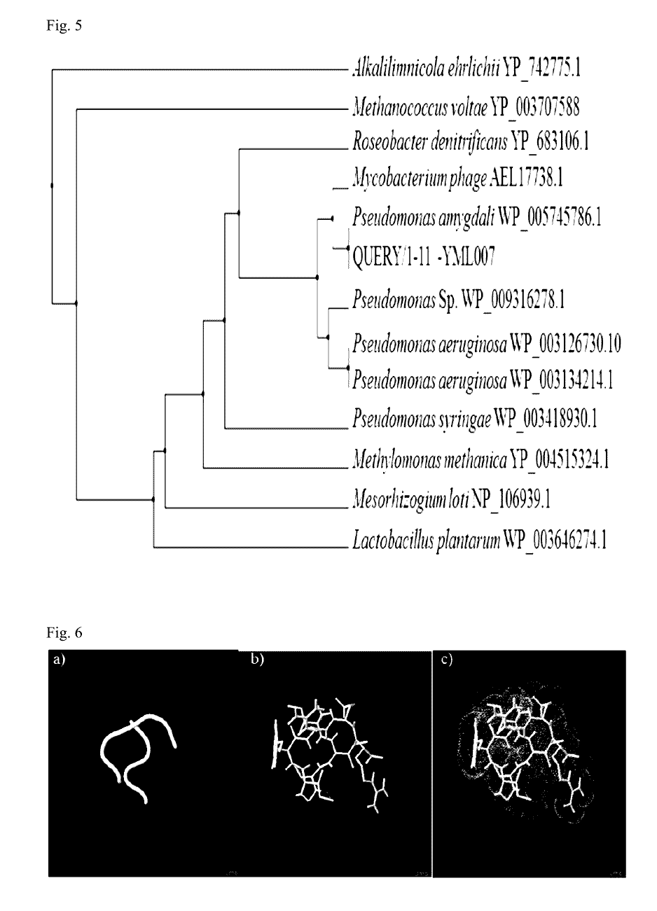

The antimicrobial peptide isolated from Lactobacillus plantarum YML007 containing 11 amino acids shows different homology patterns with other proteins of high molecular weight. The sequences align more at 5′ end of the protein. The small size of antimicrobial peptide makes it difficult to predict if these matched residues are active sites. However, majority of the sequence residues at 5′ of a protein are conservative residues. Therefore, from sequence alignment, it is impossible to conclude that the sequence of antimicrobial peptide has any structural or functional relationship with other known or unknown protein sequences. In FIG. 4, dark blue shows conservative amino acid residues, light blue represents less conservative amino acid residues, whereas without color represents non-conservative amino acid residues (FIG. 4). The average distance tree was presented using percentage identity with amino acid sequences of other proteins isolated from different organisms as shown in FIG. 5.

(4) Structure Prediction

Multiple sequence alignments were generated using BLAST and MUSCLE. The multiple sequence alignments were incorporated in GOR V to improve the discrimination among secondary structures. The structure prediction was done by GOR V protein secondary structure prediction server. The GOR (Garnier-Osguthorpe-Robson) method uses both information theory and Bayesian statistics for predicting the secondary structure of proteins (FIG. 6).

GOR V prediction (http://gor.bb.iastate.edu/)

IIVVGGIYRER

CEEEEEEECCC

Sequence length: 11 amino acids

Column Information:

1) Sequence index

2) Amino acid type

3) Helix probability

4) Sheet probability

5) Coil probability

6) GOR V prediction

1. I 0.067 0.178 0.755 C

2. I 0.070 0.595 0.336 E

3. V 0.075 0.802 0.123 E

4. V 0.112 0.744 0.144 E

5. G 0.112 0.505 0.382 E

6. G 0.142 0.462 0.396 E

7. I 0.195 0.520 0.286 E

8. Y 0.229 0.496 0.276 E

9. R 0.206 0.285 0.509 C

10. E 0.151 0.168 0.681 C

11. R 0.073 0.131 0.796 C

(5) Multiple Alignments Based on Antimicrobial Peptide Data Base

The antimicrobial peptide database (APD) is based on extensive literature search. It contains detailed information for 525 peptides (498 antibacterial, 115 antifungal, 28 antiviral, and 18 antitumor). (http://aps.unmc.edu/AP/main.html)

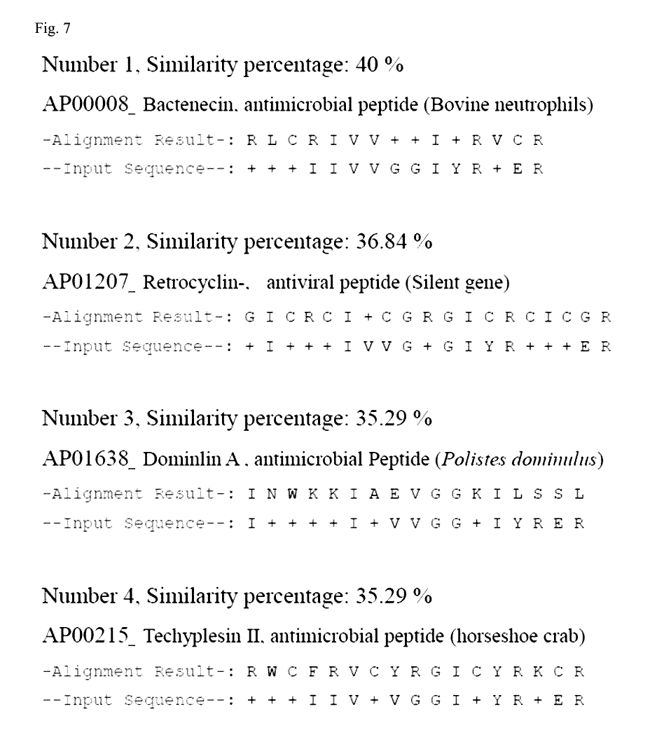

The sequence alignment of antimicrobial peptide isolated from Lactobacillus plantarum YML007 shows following percentage of similarity with other antimicrobials isolated from different sources (FIG. 7).

(6) Characterization of Antimicrobial Peptide

Sequence: IIVVGGIYRER (SEQ ID NO: 1)

I-I-V-V-G-G-I-Y-R-E-R-, where hydrophobic residues are in bold letters.

Hydrophilic amino acid—I: 3, V: 2, L: 0, F: 0, C: 0, M: 0, A: 0, W: 0,

The number of G and P—G: 2, P: 0,

Negatively charged amino acid—E: 1, D: 0,

Positively charged amino acid—K: 0, R: 2, H: 0,

Other amino acid—T: 0, S: 0, Y: 1, Q: 0, N: 0

The Ile ratio=27%, The Val ratio=18%, The Leu ratio=0%,

The Phe ratio=0%, The Cys ratio=0%, The Met ratio=0%,

The Ala ratio=0%, The Trp ratio=0%, The Gly ratio=18%,

The Pro ratio=0%, The Thr ratio=0%, The Ser ratio=0%,

The Tyr ratio=9%, The Gln ratio=0%, The Asn ratio=0%,

The Glu ratio=9%, The Asp ratio=0%, The His ratio=0%,

The Lys ratio=0%, The Arg ratio=18%

The APD defined total hydrophobic ratio=45%

The total net charge=+1

The Wimley-White whole-residue hydrophobicity of the peptide (i.e. the sum of whole-residue free energy of transfer of the peptide from water to POPC interface)=1.93 kcal/mol

The molecular weight of the input peptide=1274.533

Assuming cysteins are paired, the molar extinction coefficient of the peptide=1490

When the cysteines are not paired (e.g. reduced HBD-1), the molar extinction coefficient of the peptide=1490[190]

Protein-binding potential (Boman index): 1.09 kcal/mol

The molecular formula of the peptide: C₅₈H₉₉N₁₇O₁₅S₀

Although the present invention has been described by way of a detailed description in which various embodiments and aspects of the invention have been described, it will be seen by one skilled in the art that the full scope of this invention is not limited to the examples presented herein. Therefore, the substantial scope of the present invention will be defined by the accompanying claims and equivalents thereof.

### INDUSTRIAL APPLICABILITY

The peptide having a fungicidal activity, isolated from Lactobacillus plantarum YML007, according to an embodiment of the present invention, has a wide range of fungicidal activities, and thus can be applied, by using the same, to various fields such as a microbial preparation related to fungicidal activity, an agent for controlling pathogenic fungi, a preservative for food including feed, and the future development of medical supplies related to fungi.

1. A fungicidal peptide comprising an amino acid sequence of SEQ ID NO: 1.

2. The fungicidal peptide of claim 1, wherein the peptide is derived from Lactobacillus plantarum YML007(KCTC 12032BP).

3. The fungicidal peptide of claim 1, wherein the fungicidal peptide is effective in controlling Aspergillus niger or Aspergillus flavus.

4. A fungicidal composition comprising the peptide of claim 1 as an active ingredient.

5. The fungicidal composition of claim 4, wherein the fungicidal composition is effective in controlling Aspergillus niger or Aspergillus flavus.

6. A composition for food preservation comprising the peptide of claim 1 as an active component.
